(19) United States
(12) Patent Application Publication
(10) Pub. No.: US 2016/0003815 A1
(43) Pub. Date: Jan. 7, 2016

(54) METHOD, SYSTEM, AND DEVICE FOR ANALYTE DETECTION AND MEASUREMENT USING LONGITUDINAL ASSAY

(71) Applicant: Inanovate, Inc., Research Triangle Park, NC (US)

(72) Inventors: James Curtis NELSON, Raleigh, NC (US); David Justin SLOAN, Apex, NC (US); David URE, Wellesley, MA (US); Gregory Allen VOTAW, Garner, NC (US); Jinlong YIN, Birmingham (GB)

(21) Appl. No.: 14/854,615

(22) Filed: Sep. 15, 2015

Related U.S. Application Data
(63) Continuation of application No. PCT/US2014/024396, filed on Mar. 12, 2014.
Application No. PCT/US2014/024396 is a continuation of application No. PCT/US2014/024415, filed on Mar. 12, 2014.
Application No. PCT/US2014/024415 is a continuation of application No. PCT/US2014/024429, filed on Mar. 12, 2014.
(60) Provisional application No. 61/800,101, filed on Mar. 15, 2013.
(60) Provisional application No. 61/800,276, filed on Mar. 15, 2013.
(60) Provisional application No. 61/800,429, filed on Mar. 15, 2013.

(51) Int. Cl. G01N 33/543; B01L 3/00

(52) U.S. Cl. CPC G01N 33/54366; B01L 3/5027; B01L 2300/0636; B01L 2300/0816; B01L 2300/0877; B01L 2400/0487

(57) ABSTRACT

Embodiments of the invention provide methods, systems, and devices for detection and measurement of an analyte or analytes. In one embodiment, the invention provides an assay system comprising a cartridge device including: one or more reservoir portions for holding one or more liquids; and at least one assay portion for receiving the one or more liquids from the at least one reservoir portion, the at least one assay portion having a plurality of binding sites over which the one or more liquids from the one or more reservoirs can be flowed repeatedly (more than one time); and a measurement device for measuring binding of one or more analytes in the one or more liquids to the plurality of binding sites.

### CROSS-REFERENCE TO RELATED APPLICATIONS

This application is a continuation application of co-pending PCT Application No. PCT/US2014/024396, filed 12 Mar. 2014, which claims the benefit of U.S. Provisional Patent Application Ser. No. 61/800,101, filed 15 Mar. 2013, a continuation application of PCT Application No. PCT/US2014/024415, filed 12 Mar. 2014, which claims the benefit of U.S. Provisional Patent Application Ser. No. 61/800,276, filed 15 Mar. 2013, and a continuation application of PCT Application No. PCT/US2014/024429, filed 12 Mar. 2014, which claims the benefit of U.S. Provisional Patent Application Ser. No. 61/800,429, filed 15 Mar. 2013, each of which is hereby incorporated herein.

### BACKGROUND

The Life Sciences industry depends on the accurate and timely development and processing of tests known as bioassays, to discover new drugs, identify new biomarkers or measure biomarker levels for diagnosis and monitoring of diseases.

However, known assay methods suffer from a number of limitations. One such limitation is an inability to distinguish specific binding of a targeted analyte to a capture reagent within the assay from the non-specific binding of non-targeted substances, molecules, or analytes to a capture reagent within the assay. Any non-specific binding can lead to artificially high measurements and inaccurate determinations of analyte concentration. Indeed, in some cases, an analyte may not be present in a test sample, but the signal generated through the non-specific interaction results in a false positive test result.

A further serious limitation of known assay methods is their inability to accurately detect and measure different analytes that may be present within the sample(s) being analysed at concentrations that differ by many orders of magnitude. This limitation to quantitate analyte concentrations across a broad dynamic range in a single test limits the biological relevance of many multiplex assays presently available, and also leads to the need to run multiple serial dilutions of samples, requiring additional time, money and the use of precious sample.

Furthermore, most known assay methods require relatively complex, user intensive protocols, limiting the accessibility of many assays to only well equiped laboratories with trained and skilled staff.

International Patent Application Publication No. WO 2012/071044 (“the '044 application”), which is hereby incorporated herein as though fully set forth, describes longitudinal assay methods that overcome many of the limitations of known assays; and the system and methods disclosed herein further address the limitations set out above and provide a unique and novel approach to the accurate detection and measurement of multiple analytes.

### SUMMARY

The invention herein disclosed describes a system and associated methods incorporating and building from longitudinal assay screening.

Central to the system and methods disclosed is the ability to collect image data during the course of a bioassay incubation process. This enables the collection of time-course or longitudinal data used in the generation of real-time kinetic binding curves.

In one embodiment, the invention provides for an assay system comprising: a cartridge device including: one or more reservoir portions for holding one or more liquids; and at least one assay portion for receiving the one or more liquids from the at least one reservoir portion, the at least one assay portion having a plurality of binding sites over which the one or more liquids from the one or more reservoirs can be flowed repeatedly (more than one time); a measurement device for measuring binding of one or more analytes in the one or more liquids to the plurality of binding sites.

In another embodiment, the invention provides for a system comprising: a receptacle for receiving a fluidic cartridge with an interface for controlling flow rates of one or more fluids from one or more reservoirs in said cartridge; and a fluidic cartridge with one or more reservoirs containing fluids at least one of which is a fluid to be analyzed, which may contain a targeted analyte or analytes, and at least one fluid contains a fluorescent, luminescent or colormetric label, and said cartridge also containing fluidic channels capable of moving one or more fluids under computer control from one section of the cartridge to another, at least one such section, the assay portion of the cartridge, containing an array of more than one binding site, each site containing a specific capture agent or capture agents; and a device for interacting with and/or stimulating fluorescent or luminescent or colormetric labels, and measuring the intensity of the fluorescent or luminescent or colormetric signal at each such site in time sequence; and an apparatus for assimilating such time sequenced measurements to create a representation of a dynamic or kinetic binding curve between a non-targeted substance/molecule/analyte and capture agents or other substances within the binding sites.

In another embodiment, the invention provides for a method of distinguishing specific binding and non-specific binding in an assay, the method comprising: obtaining a plurality of signal intensity measurements of an analyzed sample, each of the plurality of signal intensity measurements being made during an iterative interaction of the analyzed sample and a capture agent for a targeted analyte within the analyzed sample; plotting the plurality of signal intensity measurements as a function of cumulative duration of interaction of the analyzed sample and the capture agent.

In still another embodiment, the invention provides for a method of calculating an analyte concentration in a sample containing the analyte, the method comprising: obtaining a plurality of signal intensity measurements representing binding of a component of the sample to a capture agent capable of binding to the analyte, the plurality of signal intensity measurements being made at known time intervals and for known durations of interaction between the sample and the capture agent; plotting the plurality of signal intensity measurements as a function of cumulative duration of interaction of the sample and the capture agent; calculating one or more first derivatives or tangents (initial rates) to the plotted signal intensity measurements, each of the one or more first derivatives or tangents (initial rates) being calculated using adjacently-plotted signal intensity measurements; the first derivatives or tangents (initial rates) for the one or more standard analyte concentrations can subsequently be used to determine the back calculation curve fit. The back calculation fit is a fit based on an enzyme catalyst model, where an enzyme substrate complex is formed prior to reaction. The unknown sample first derivative or tangent (initial rate) is then used to back calculate the concentration from the one or more standard analysis concentrations.

In still another embodiment, the invention provides for a cartridge device comprising: at least one reservoir portion for holding one or more fluids; and at least one assay portion for receiving the one or more fluids from the at least one reservoir portion, the at least one assay portion having a plurality of binding sites over which the fluid is flowed, and being connected to the at least one reservoir portion through fluidic channels or tubing.

### BRIEF DESCRIPTION OF THE DRAWINGS

These and other features of the invention will be more readily understood from the following detailed description of the various aspects and embodiments of the invention taken in conjunction with the accompanying drawings that depict various embodiments of the invention, in which:

FIG. 1 shows an exploded perspective view of an assay cartridge according to an embodiment of the invention.

FIG. 2 shows a simplified schematic view of four assay portions of an assay cartridge according to an embodiment of the invention.

FIG. 3 shows a simplified schematic view of a single assay portion of an assay cartridge, plus simplified schematics for the reservoirs and valving components of the system and assay cartridge according to an embodiment of the invention.

FIGS. 4-7 show simplified views of various steps of an assay method according to an embodiment of the invention.

FIGS. 8-10 show simplified views of analyte-capture agent and accelerator interactions according to another embodiment of the invention.

FIG. 11 shows a graph of signal intensities of targeted and non-targeted analyte bindings.

FIGS. 12-14 show binding curves of a triplex assay according to an embodiment of the invention targeting, respectively, IL-6, CRP, and IL-1b.

It is noted that the drawings are not to scale and are intended to depict only typical aspects of the invention. The drawings should not, therefore, be considered as limiting the scope of the invention. In the drawings, like numbering represents like elements among the drawings.

### DETAILED DESCRIPTION

### Definitions

As used herein, various terms and phrases are intended to have meanings within the context of the claimed invention, as will now be described. The term “fluid” is meant to broadly encompass states of matter subject to or capable of flowable movement, and includes liquids, gasses, and fine particulate solids, as well as combinations and suspensions thereof, such as colloidal suspensions. In the context of the present invention, a “liquid” may include, but is not limited to, animal (including but not limited to human) blood, serum, saliva, urine, plasma, bronchial alveolar lavage, bronchial lavage, tissue, tumors, and tissue/tumor homogenates, as well as plant extract, liquified food matter, ascites fluid, organic fluids, inorganic fluids, buffers, labeled buffers, washes, etc.

The term “analyte” is intended to mean any thing to be or capable of being detected or measured. This includes, but is not limited to, biological entities, such as proteins, hormones, antibodies, antigens, viruses, antibody complexes, peptides, cells, cell fragments, aptamers, cell lystates, DNA, RNA, mRNA, genes, genetic expression products, etc., as well as chemical entities, such as chemical elements, chemical compounds, pharmaceutically-active compounds or their metabolites, minerals, pollutants, etc.

The term “detector reagent” (or detection reagent) is intended to mean anything used as a mechanism for enabling the visualization (detection) and/or measurement of the analyte. This includes, but is not limited to, biological entities, such as proteins, hormones, antibodies, antigens, viruses, antibody complexes, antibody fragments, peptides, cells, cell fragments, aptamers, cell lystates, DNA, RNA, mRNA, genes, genetic expression products, etc., as well as chemical entities, such as chemical elements, chemical compounds, pharmaceutically-active compounds or their metabolites, minerals, pollutants, etc.

Detector reagents may also be labeled with fluorescent, luminescent or colorimetric labels in order to facilitate visualization (detection) and/or measurement, and they may be further conjugated with an affinity tag label such as biotin. This would allow the use of a fluorescent, luminescent, or colorimetrically labeled streptavidin for the visualization (detection) and measurement of the detection antibody.

The terms “detect” and “measure,” as well as their variants, are meant to refer, respectively, to the identification of the presence of a thing and an assessment of a variable feature of a thing, such as concentration, strength, intensity, etc. The term “analyze” and its variants, refers more broadly to such detection and measurement, as well as to other or different assessments of an event.

The phrase “binding site” is meant to refer to a locus at which an interaction with an analyte is possible or intended and at which detection or measurement may be made. Typically, a binding site will include a capture agent. The phrase “capture agent” (or capture reagent), in turn, is meant to refer to any structure, compound, system, or device with which an analyte may interact. Often, such interaction will include structural or chemical interaction, although this is not essential. Capture agents include, but are not limited to, biological entities, such as proteins, hormones, antibodies, antigens, viruses, antibody complexes, antibody fragments, peptides, cells, cell fragments, aptamers, cell lystates, DNA, RNA, mRNA, genes, genetic expression products, etc., as well as chemical entities, such as chemical elements, chemical compounds, pharmaceutically-active compounds or their metabolites, minerals, pollutants, etc.

The phrase “signal intensity” is meant to refer to a measurement of interaction at a binding site, such as by interaction of a capture agent and an analyte. Such measurement may be made directly or indirectly and by any number or combination of techniques, such as fluorescence, luminescence, or colorimetric labeling.

The phrase “standard binding curve” is meant to refer to a binding curve of the binding interaction of a capture agent and analyte at known concentrations and against which the binding interactions of the capture agent and analyte at unknown concentrations may be compared. The phrases “representation of a dynamic binding curve” and “representation of a kinetic binding curve” are meant to refer to representations of the dynamics or kinetics of binding interactions of a capture agent and analyte. These are distinct from a typical and more conventional end-point standard curves that are representations of the signal intensity from assays at given analyte dilutions at a specific time point (i.e. on completion of the assay). These may include data obtained from all or portions of the steps of a binding assay and may be used to determine whether a detected or measured interaction and/or a corresponding signal intensity is attributable to a specific binding interaction. A “binding curve” or “kinetic curve” can describe a signal growing from low to high due to the exposure of binding sites to samples and detector reagents (association) as well as a signal decreasing from high to low once sample and detector reagents are not present (disassociation).

### Assay System or Platform

Components of an assay system according to some embodiments of the present invention are described in the '044 application. These include, for example, devices and apparatuses for controlling movement of a liquid across the binding sites. These may include, for example, a pump or a vacuum device capable of exerting a positive or negative pressure, respectively, on a liquid. In other cases, the device or apparatus may include a capillary or similar structure through which the liquid may be moved via capillary action. In any case, such devices and apparatuses allow one or more aspects of the flow of the liquid to be controlled, such as a rate of flow of the liquid, a duration of flow of the liquid, or the number of times a quantity of the liquid is flowed over the binding sites.

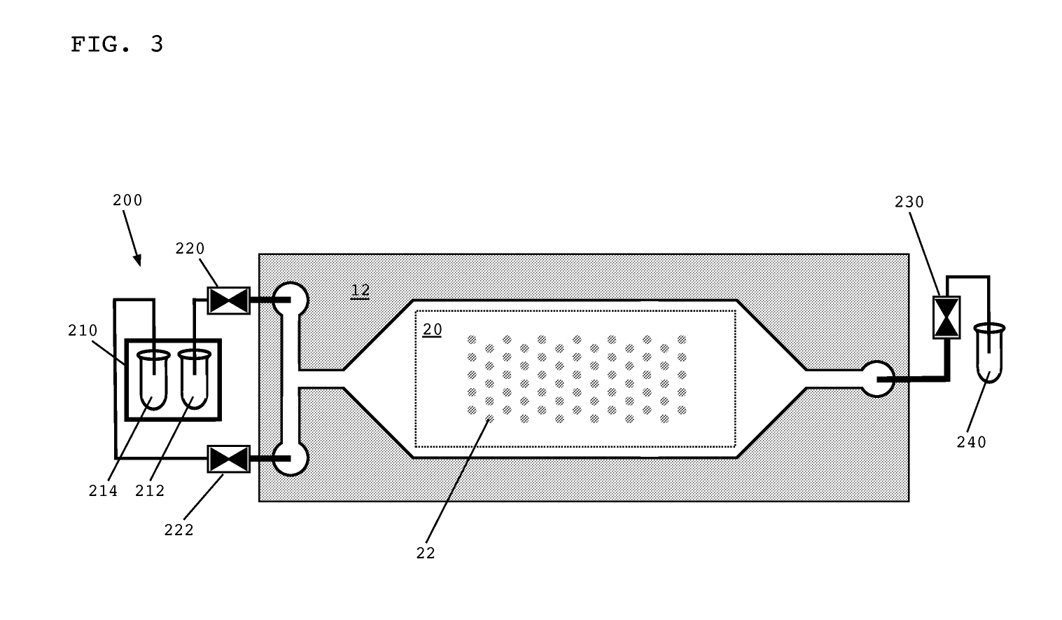

As noted above and will be described in more detail below, some embodiments of the assay system according to the invention utilize a cartridge for both containing the sample and supplies of other assay reagents as well as an area in which the assay itself is carried out. Accordingly, some embodiments of such cartridges will include one or more reservoir portions for holding the sample and/or assay reagents and a separate assay portion into and through which the sample and reagents may be flowed. As will be described with respect to FIG. 3, the assay portion includes a plurality of binding sites (containing the capture agents) over which the liquid is flowed and at which the target analyte and capture agents interact.

The assay portion of the cartridge includes at least one location at which binding of the target analyte and capture agent may be detected and/or measured, and more typically there will be multiple locations, such as printed spots (or dots) of capture agents. Each printed spot may contain one or more types of capture agent, with each printed spot in the assay portion containing the same or different types of apture agent. In some embodiments of the invention, the assay portion of the cartridge will include a transparent surface—often a glass—through which detection/measurement of the binding events may be made. In the case that the assay system employs a fluorescent detection device, an excitation beam may be passed through the transparent surface onto the binding sites to excite a fluorescently-labeled analyte or analyte-capture agent complex. Emission by the fluorescently-labeled analyte or analyte-capture agent complex may then be detected/measured through the same or a different transparent surface.

In certain embodiments, the system incorporates a scanner that uses a confocal approach in which a 532 nm laser beam is focused and brought incident onto the surface of the assay portion of the cartridge.

The imaging is accomplished through the base of the cartridge from underneath the assay solution. The assay surface contains printed capture agents (typically proteins or antibodies, but in some embodiments may be other biological entities, such as hormones, viruses, antibody complexes, antibody fragments, peptides, cells, cell fragments, aptamers, cell lystates, DNA, RNA, mRNA, genes, genetic expression products, etc., as well as chemical entities, such as chemical elements, chemical compounds, pharmaceutically-active compounds or their metabolites, minerals, pollutants, etc.) which are used in a fluorescently labeled sandwich-type binding assay to quantitatively measure analyte concentrations.

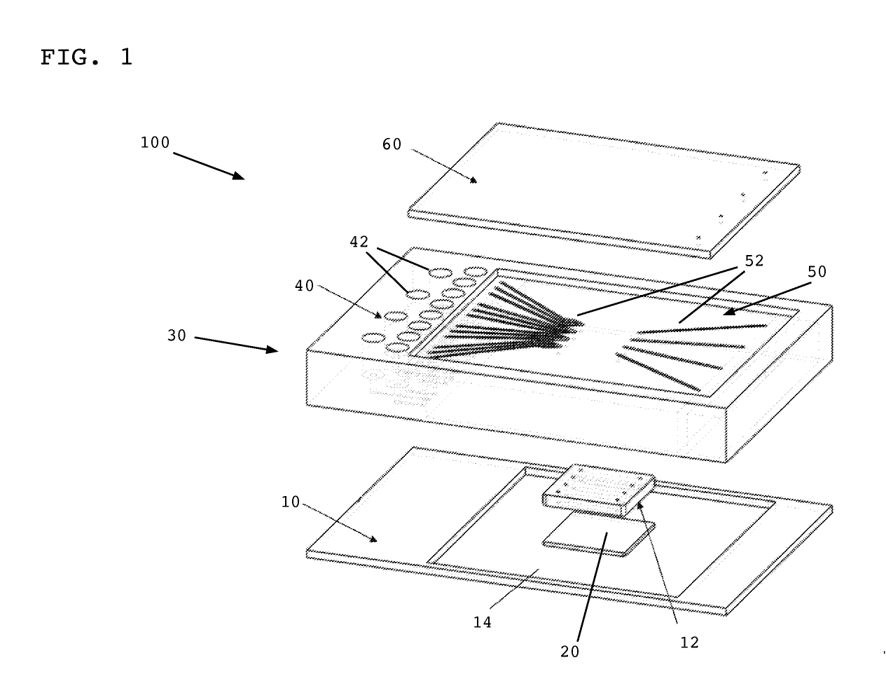

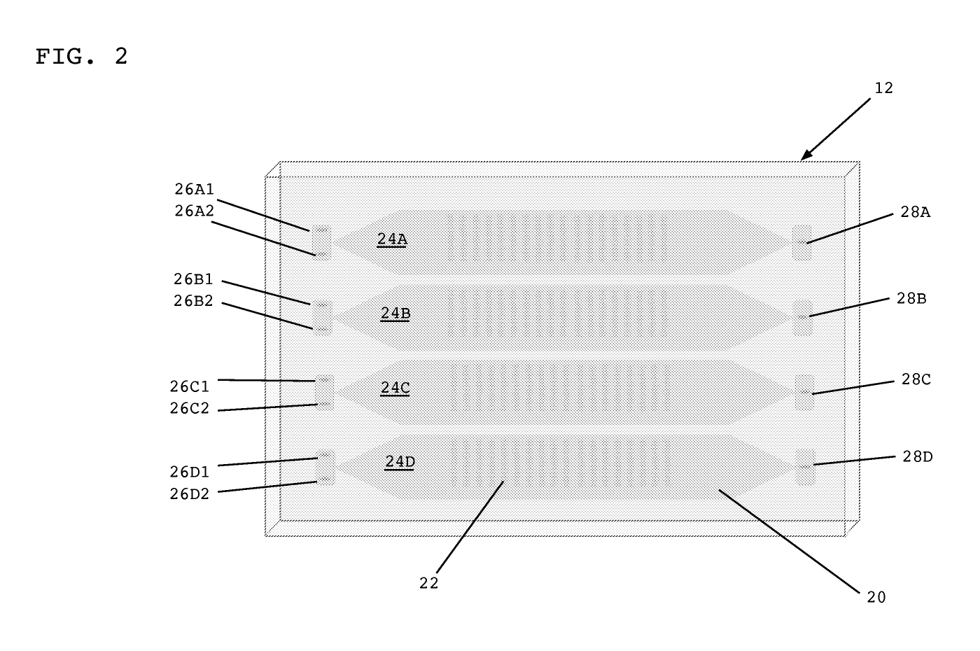

The fluorescent light emitted from the assay on the cartridge surface is collected back through the excitation optics path and diverted onto a photomultiplier tube (PMT) through a series of spatial filters and mirrors. The “scanning” in the system is accomplished via a dual axis galvometer driven mirror assembly and telecentric lens system capable of generating high resolution images that are pixel aligned with time, although other mirror and lens systems may be used. In certain embodiments the allowed scan area for the system is 25×25 mm in other embodiments this may be reduced or increased to 100 mm×100 mm or above, however in these and other embodiments the assay portion of the cartridge may be smaller than the allowed scan area, and range from 1 mm×1 mm through 100 mm×100 mm and above. In some embodiments there are 4 assay portions available per cartridge. See, e.g., FIGS. 1 and 2. However, in other embodiments, the number of assay portions may range from 1 through 100.

The fluorescent laser scans the assay through the underside of the cartridge as samples are iteratively flowed across the assay; enabling time-course binding data to be collected and compiled into a high sensitivity kinetic binding curve. The binding curves of each assay on the cartridge are processed by analysis software, and data on analyte presence and concentrations delivered to users.

### System Components

### 1. Detector

In certain embodiments the detector may comprise an excitation light path and an emissions light path that combine to encompass the detector. The excitation light path may comprise of a light source, in certain embodiments this may be a 532 nm laser, conditioned via a beam expander and aperture assembly that collimates and sizes the laser light source.

In certain embodiments, the source may then be guided to the sample by way of a dichroic beam splitter, focusing lens assemble, an X-Y axis galvometer driven mirror assembly and a telocentric lens. The galvometer driven mirror and telocentric lens assembly enable fixed mounting of the sample target thus eliminating the requirement to align multiple images in post processing. Fixed mounting of the sample also enables the coupling of the flow control module to the sample cartridge and eliminates noise in the fluidic control system typically introduced by vibrations from moving high precision fluidics systems.

In certain embodiments, the detection light path is comprised of a detector (for example a photomultiplier tube (PMT)), a band pass filter, pinhole aperture, focusing lens, dichroic beam splitter and telocentric lens. In such embodiments, once a sample has been excited by the light source it emits a photon that is collected by the telocentric lens and guided back through the excitation light path to the dichroic beam splitter. The beam splitter allows the emission wavelength to pass through to the focusing lens and is focused to a point and passed through the pinhole aperture. The pinhole aperture eliminates any out of focus light and therefore makes the detector confocal in nature. The focused light may then be passed through the band pass filter allowing only the wavelength of interest to interact with the PMT. The PMT intensity value may then be recorded as a single pixel and spatially assigned based on the x-y position of the galvometer controlled mirrors. In this way an image may be produced that includes the pixel position and intensity.

### 2. Flow Control Module

The system may incorporate a microfluidic controller for control of the flow of liquids through multiple assay portions of the cartridge (assay cells). In embodiments wherein the assay cartridge contains four (4) such assay portions (or assay cells—see FIG. 2), the microfluidic controller may include a computer controlled pressure regulator, 3-way valve, pressurized sample vessel, 8 inlet valves, 4 outlet valves and a micro-manifold that in certain embodiments may direct flow to a precision flow sensor from the 4 outlet valves. The controller may then enable precise timing of flow events down to milliseconds, as well as slower pre/post assay operations. The dynamically controlled regulator (at the beginning of the flow path) coupled with a precision flow sensor (at the end of the flow path) may work in tandem to create a closed loop flow control system that ensures accurate flow rates and volumes regardless of flow path characteristics. The controller also provides real-time feedback for all valve states to the user. The Flow Control Module may optionally be built into the body of the system; and designed such that it allows users to efficiently insert and replace samples and reagents. In certain embodiments the flow control module may have more or less than 8 inlet and 4 outlet valves, the number of valves being proportional to the number of assay portions included on the cartridge, e.g. in certain embodiments, each assay portion requires 2 inlet and 1 outlet valve (refer to FIG. 3).

### 3. Assay Cartridge

In some embodiments, such as that shown in FIG. 1, the assay cartridge 100 includes a treated glass surface 20 printed with capture agents (typically proteins or antibodies, but in some embodiments may be other biological entities, such as hormones, viruses, antibody complexes, antibody fragments, peptides, cells, cell fragments, aptamers, cell lystates, DNA, RNA, mRNA, genes, genetic expression products, etc., as well as chemical entities, such as chemical elements, chemical compounds, pharmaceutically-active compounds or their metabolites, minerals, pollutants, etc.) and bonded to a Polydimethylsiloxane (PDMS) block 12 molded to contain flow channels and assay portions (assay cells), as will be described in greater detail below. PDMS is only one material that may be employed, of course, and should not be viewed as limiting the scope of the invention. Other suitable materials include, for example, chemically treated glass, glass covered with nano-particles, glass coated with metals, glass treated with other forms of materials, carbon (all forms), silicon, rubber, plastic, metals, crystals, polymers, semi-conductors, organic materials and in-organic materials.

In some embodiments, such as that shown in FIG. 1, assay cartridge 100 further includes a bottom plate 10 having a transparent portion 14 through which detection/measurement of analyte-capture agent interactions may be made, a body 30 for interfacing the assay reservoirs with the PDMS block 12 and treated glass surface 20, and optionally a top plate 60 atop the body 30. Treated glass is only one material that may be employed for surface 20, and should not be viewed as limiting the scope of the invention. Other suitable materials include, for example, PDMS, glass covered with nano-particles, glass coated with metals, glass treated with other forms of materials, carbon (all forms), silicon, rubber, plastic, metals, crystals, polymers, semi-conductors, organic materials and in-organic materials.

Body 30 may include a reservoir portion 40 having a plurality of reservoirs 42 for holding a test sample and/or assay reagents (including detector reagents and optionally accelerators). Body 30 may further include a transport area 50 having a plurality of channels 52, through which quantities of test sample and assay reagents can be flowed to and from the PDMS block 12 and treated glass surface 20.

In certain embodiments, the body 30 and the PDMS block 12 may be replaced with one integrated body/block which is molded or otherwise constructed out of PDMS or other suitable materials such as for example, chemically treated glass, glass covered with nano-particles, glass coated with metals, glass treated with other forms of materials, carbon (all forms), silicon, rubber, plastic, metals, crystals, polymers, semi-conductors, organic materials and in-organic materials. In such embodiments as well as other embodiments, the bottom plate 10, transparent portion 14 and treated glass surface portion 20 may all be replaced with one surface which may be treated glass, or in certain embodiments may be glass covered with nano-particles, glass coated with metals, glass treated with other forms of materials, carbon (all forms), silicon, rubber, plastic, metals, crystals, polymers, semi-conductors, organic materials and in-organic materials.

In yet further embodiments, the reservoirs may be separated from the assay cartridge, and connect to the PDMS block 30 (or other such material as described above) through connecting tubing. In such embodiments only the PDMS block 30 (or other such material as described above) and the treated glass surface 20 (or other such material as described above) are required to form the assay cartridge.

FIG. 2 shows a detailed view of the PDMS block 12 and treated glass surface 20 of FIG. 1 and other embodiments as described above. In this embodiment there are 4 assay portions contained in the assay cartridge. Here, the plurality of binding sites 22 (or capture spots or dots) on treated glass surface 20 may more easily be seen. In some embodiments, such as that shown in FIGS. 1 and 2, each assay portion 24A, 24B, 24C, 24D has two inlets 26A1-2, 26B1-2, 26C1-2, 26D1-2 and one outlet 28A, 28B, 28C, 28D, allowing the rapid interchange of multiple assay reagents, as will be described further below.

As previously noted, treated glass is only one material that may be employed for surface 20, and should not be viewed as limiting the scope of the invention. Other suitable materials include, for example, PDMS, glass covered with nano-particles, glass coated with metals, glass treated with other forms of materials, carbon (all forms), silicon, rubber, plastic, metals, crystals, polymers, semi-conductors, organic materials and in-organic materials.

As previously noted, in certain embodiments, such as that shown in FIGS. 1 and 2, the cartridge may contain 4 such assay portions (cells), each allowing many hundreds of binding sites (capture spots) per cell (the number of spots per cell can range from 2 to over 20,000). Binding sites or capture spots are herein used to describe areas on the surface which contain groups of capture agents. Such capture spots may be placed on the treated glass surface 20 using a micro-array printer device or through any alternative method, as will be apparent to one skilled in the art. In some embodiments, the assay cartridge may be connected to the reservoirs and through these to the flow control system using peek tubing (connector tubing) via a multi-pin connection manifold.

FIG. 3, for example, shows a simplified view of a cartridge according to an embodiment of the invention in conjunction with various assay reagents as part of a fluid control system 200. For purposes of simplicity, only PDMS block 12 and treated glass surface 20 of the cartridge is shown.

Here, a test sample 212 and assay reagents 214 are contained within pressurized reservoirs (input wells) 210. Upon opening a first input valve 220, the test sample 212 is introduced to the assay portion (comprising PDMS block 12 and treated glass surface 20) of the cartridge, such that the test sample flows over binding sites (capture spots) 22. Opening outlet valve 230 permits test sample 212 to then pass to a waste chamber 240.

An assay reagent 214 may then be introduced to the assay portion by opening inlet valve 222, allowing assay reagent 214 to flow over binding sites (capture spots) 22. Assay reagents 214 may then be evacuated to waste chamber 240, as described above. This iterative flow process may then be repeated multiple times.

In certain embodiments following existing the assay portion, both sample 212 and assay reagents 214 are first flowed across a precision flow sensor before being evacuated into a waste chamber 240. The controller enables precise timing of flow events down to milliseconds, as well as slower pre/post assay operations. The dynamically controlled regulator (at the beginning of the flow path) coupled with a precision flow sensor (at the end of the flow path) work in tandem to create a closed loop flow control system that ensures accurate flow rates and volumes regardless of flow path characteristics.

One skilled in the art will recognize, of course, that embodiments of the invention may include a plurality of test samples and assay reagents. The embodiment depicted in FIG. 3 is merely for purposes of illustration.

In other embodiments, the cartridge may incorporate passive valves, wherein the liquids (sample, labeled detector antibodies, buffer solutions etc. . . . ) are placed into reservoirs on the cartridge itself and the flow of liquid from the reservoirs through the assay portion of the cartridge may be controlled by regulating the pressure through an interface built into the system that attaches to the disposable cartridges.

### System Methods and Procedures

### 1. Assay Process

In one embodiment, the overall processing time for each cartridge and associated assays can be from 2 to 200 minutes, depending on the concentration of the analytes being detected and measured. Once the sample is primed, processing of the sample(s) across the assay(s) and associate data collection/processing, may be fully automated. Users may simply select which assay protocol to use (via the systems software interface), the system may then automatically run the assay and collect and process all associated data.

An example process is detailed in FIGS. 4-7. FIG. 4 shows a schematic view of an assay portion of an assay cartridge, which includes a plurality of binding sites (capture spots) 22, each including a plurality of capture agents 23. Some embodiments of the invention may include a plurality of capture spots containing different capture agents. In other embodiments, each capture spot 22 may include the same (only one type of) capture agent, and different capture spots may contain different or the same capture agents such that in the assay portion of the cartridge there are a plurality of capture spots, each containing one type of capture agent with many different capture agents represented by many different capture spots across the surface.

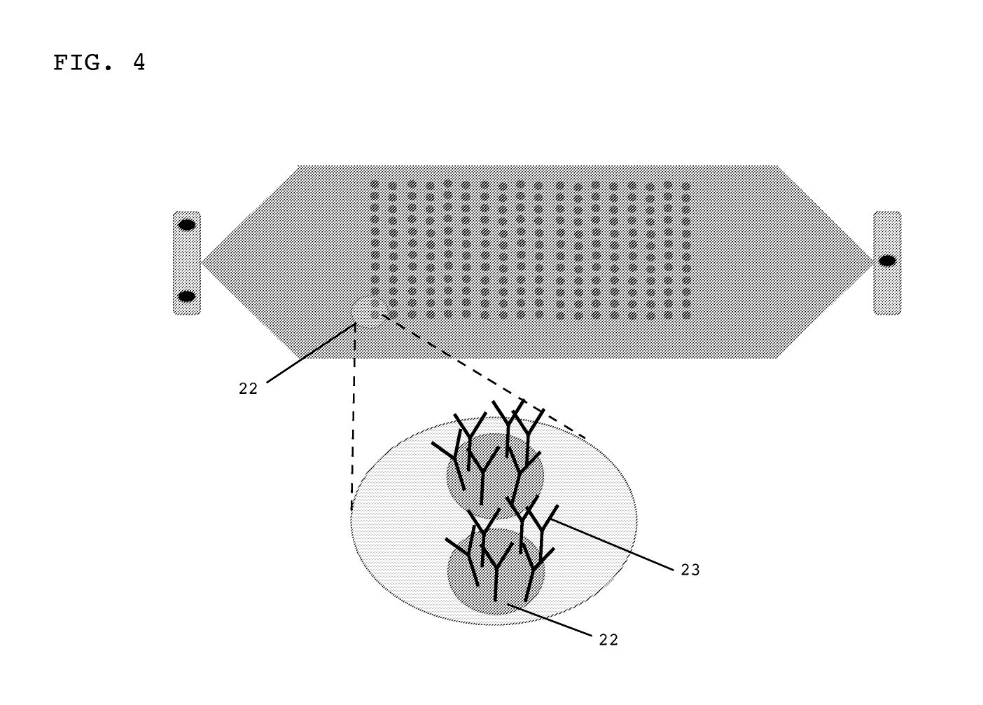

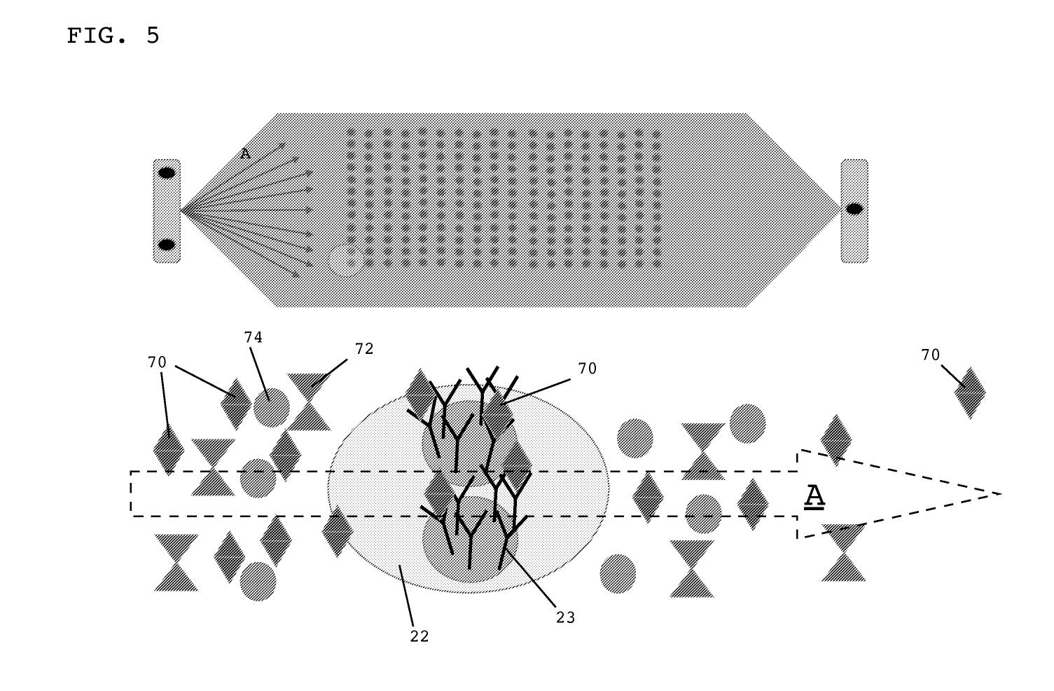

Once the assay portion of the cartridge surface is appropriately printed with capture agents 23 and blocked, the sample is flowed A across the assay portion of the cartridge, as depicted in FIG. 5. If the sample contains the target analyte (analyte of interest) 70, that analyte 70 will bind to the immobilized capture agents 23 while other analytes 72, 74 within the sample will flow across and through the assay portion of the cartridge without binding.

As shown in FIG. 6, after a defined volume of sample is flowed for a defined period of time (optionally controlled through computer software), the detection reagent 71 will be flowed B through the assay portion of the cartridge (assay chamber). The detection reagent 71 flushes the sample from the chamber and binds specifically to any analytes 70 that have been immobilized by the capture agents 23. After a defined volume of detection reagent is flowed for a defined period of time, the sample may once again be flowed through the assay portion (chamber), as depicted in FIG. 5, with these steps iteratively looped any number of times. The binding sites (capture spots) are imaged (visualized) through the measurement apparatus during these iterative loops to construct a representation of a kinetic or dynamic binding curve of target analyte to capture agent.

In some embodiments of the invention, such as that shown in FIG. 7, the sample may also contain fluorescently labeled streptavidin 76 or a similar compound. In such an embodiment, the fluorescently labeled streptavidin 76 binds to the detection agents 23, which are biotinylated, and produces the signal that can be measured and used to quantify the level of targeted analyte(s) 70 present in the sample. Additionally, in such an embodiment, more analyte of interest (targeted analyte(s)) 70 can bind to the immobilized capture agent 23, providing additional sites in subsequent iterative flow cycles for detection reagent binding.

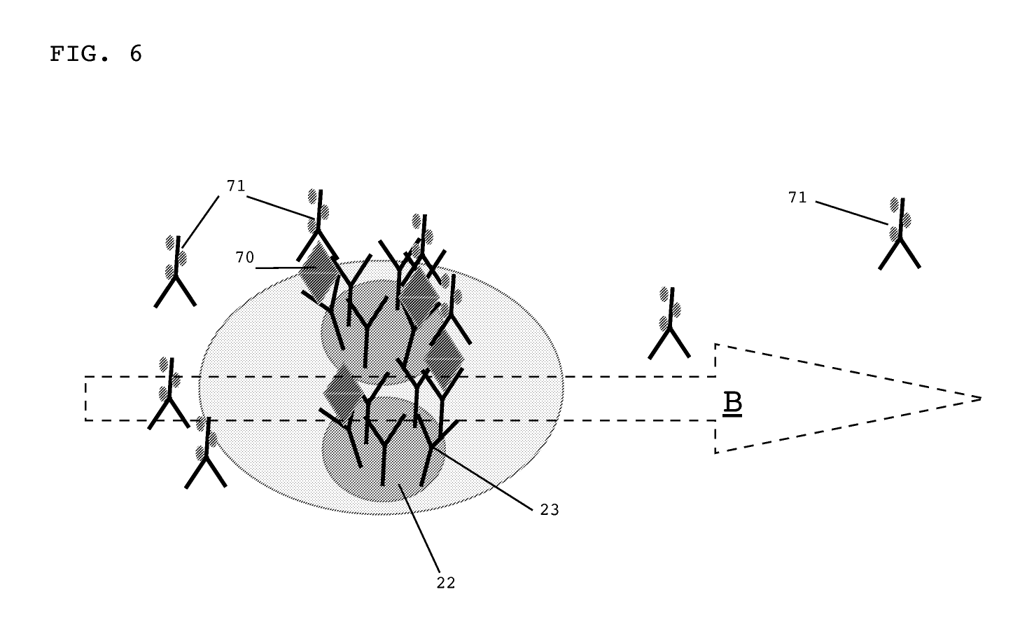

FIGS. 8-10 show steps of another process according to the invention, by which the signal can be amplified. In FIG. 8, fluorescently labeled streptavidin 76 binds to the biotinylated capture agent 71. Streptavidin is tetravalent, and can bind 4 biotins. Also present in the detection reagent cocktail is a biotinylated dendrimer 78, which contains between 1 and 8 biotins/dendrimer, and can bind to the fluorescently labeled streptavidin 76.

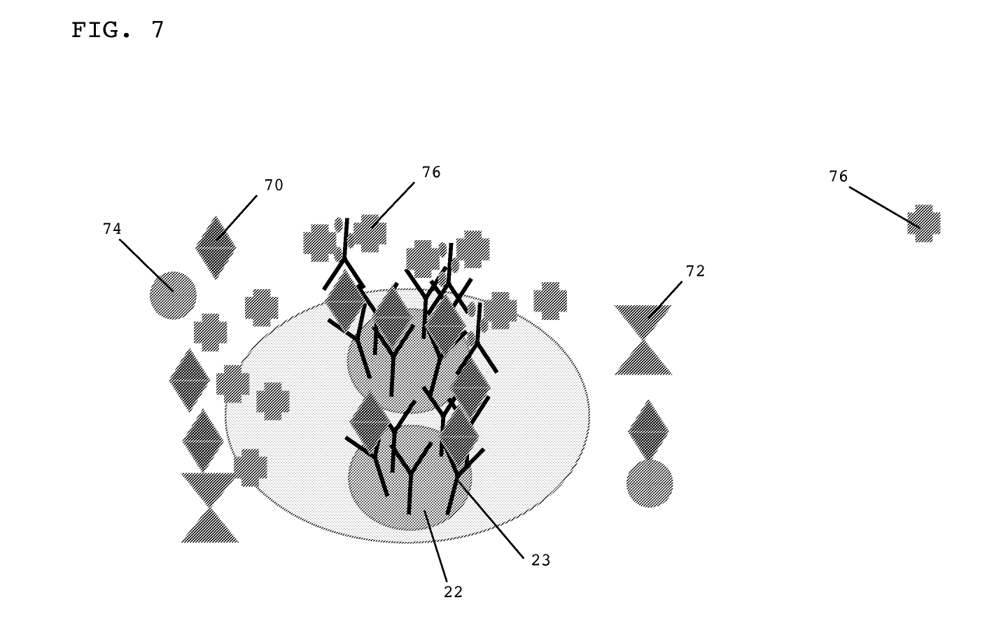

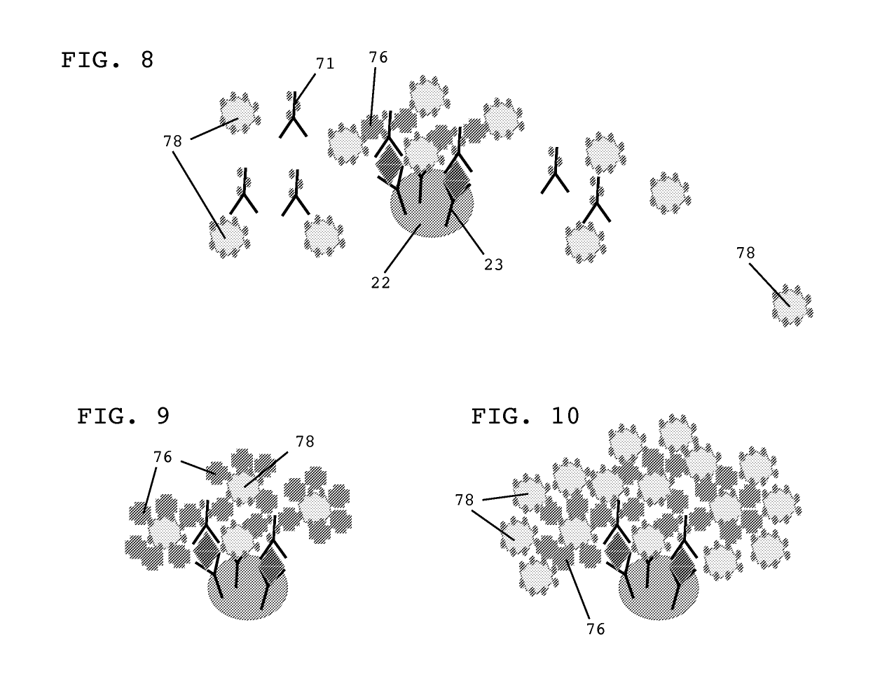

FIGS. 9 and 10 show additional fluorescently labeled streptavidins binding to the biotinylated dendrimer, causing a large increase in signal and a greatly increased sensitivity and ability to detect very low levels of targeted analyte(s) 70. As the reaction proceeds, multiple layers of fluorescently labeled streptavidin and biotinylated dendrimer can accumulate on the capture spots in an analyte dependent manner (i.e. directly correlated to the concentration of the targeted analyte(s) 70 in the sample).

While this description and the associated figures walk through the entire process as if in discrete steps, in reality there are only two steps to the reaction. In the first step, sample containing the analyte of interest and fluorescently labeled streptavidin are flowed across the surface. The analyte of interest binds to immobilized capture reagents, and fluorescently labeled streptavidin will bind to any biotinylated detection reagents or dendrimers which are present on the surface. In the second step of the reaction, biotinylated detection reagent and biotinylated dendrimer are flowed across the assay surface. If there are immobilized analytes, the biotinylated detection reagent will bind to those analytes and if there is immobilized streptavidin, the biotinylated dendrimer will bind to the immobilized streptavidin. These two steps are repeated for a defined number of cycles, with the assay surface being visualized (imaged) at the completion of each step within all cycles. This assay paradigm results in a highly specific and very sensitive sandwich immunoassay capable of specifically detecting very low levels of analyte.

### 2. Running Assays

In certain embodiments of running assays on the system disclosed herein, typical design factors are assay type, assay reagents and concentrations, the number of assays per assay portion of the cartridge, and the design of the microarray (the pattern of the capture agents on the surface). Each assay portion on a cartridge can be prepared with the same or different assay(s). Additionally, each assay portion (or cell) can run a separate experiment or sample. An example experimental design is provided below, however it should be noted that this is for example purposes only and other experimental designs varying the time, sequence and/or presence of some or more of the parameters included below may also be used and are incorporated by reference herein.

Initial System Test:

This is a short 2-15 minute beginning of the day process that is run to flush the system with flushing buffer and verify system performance. A “Test device” is connected to the system from the previous day. The user collects and measures flow volumes to verify system performance. Typically, flow variation is around 1-2% CV with a 5% to 10% pass/fail criteria. Although these criteria may optionally be set as the user wishes and/or as is appropriate for any given assay or experiment. The system flow test may be performed automatically by the computer controlled flow control module.

Device Connection and Blocking:

The Test device is removed and a user specified assay cartridge is placed on the stage of the system herein disclosed, and a connection manifold may connect the flow tubing to the cartridge ports (e.g. 8 inlets and 4 outlets for cartridges containing four assay portions, but there may be more or less). The assay portions are filled with blocking solution, forcing air out in a short (2 to 15 min) filling process. In some embodiments there is no flow tubing as the liquids (samples, reagents, detection antibodies, labels, buffers etc. . . . ) may be placed directly into reservoirs housed within the disposable assay cartridge.

Sample Prime:

The blocking solution may be replaced with sample vials and a rapid sample prime through the device may be performed to ensure that maximum sample and reagent concentration are present at the inlet of the flow device (tubing is flushed or primed where relevant).

Assay Incubation and Data Collections:

The process of flowing the sample and/or detection reagent over the assay portion for desired incubation time. After each incubation period, an imaging event is performed to capture the signal intensity due to binding of analytes to capture agents. Many such imaging events automatically occur through the assay flow process, building a binding curve from multiple time course fluorescent measurements.

System Flush:

Performed using the Test device and flushing buffer. The system flush removes assay reagents from the flow controller, prepping the system for the next assay run.

### 3. Data Collection and Analysis (for Certain System Embodiments)

Raw Data:

In certain embodiments, in its rawest form, data may be collected as a 16-bit grayscale tiff image. Each pixel in the image corresponds to a pre-selected image resolution. The data is collected instantaneously from the PMT at the desired time interval.

Time Course Data:

The signal intensity over time is the first level of processing that occurs. This type of data can be derived using several methods (user selected). In all cases it is a variation of a signal intensity number over time for each assay.

Calculation of Analyte Concentrations:

Known concentration standards are used to generate assay specific, time-course standard curves. These known standards alongside rate equations disclosed herein and accompanying proprietary algorithms are then used to analyze the kinetic curves of each user-processed assay, to deliver a precise measurement of the concentration of each target analyte.

Statistical Confidence:

This is a threshold confidence level relating the sample curve to the expected curve. Once the confidence of the unknown curve (across all assays in a multi-plea) is met the assay incubation process can be terminated, optimizing data collection while minimizing processing time.

### 4. Detailed Analysis Procedures

Analysis of the data generated from the system and methods herein disclosed is predicated upon the measure of the initial binding rate between a capture reagent (which in some embodiments is a targeted monoclonal antibody), which is some embodiments is printed or otherwise placed in a microarray format, and an analyte of interest or target analyte (which in some embodiments is a target protein). The quantitative analysis method is based upon the measured initial binding rate and back calculation from known standards. While the initial binding rate is a delta measurement between two time point and thus not as susceptible to absolute signal intensity issues associated with variable background, the determination of limits of detection are effected by variable background trends. As such, data can be corrected by a simple subtraction of signal intensity from a low signal background region in each image and each time point.

In some embodiments, ‘Linear Data’ are produced by the system and methods herein disclosed, and analyzed as follows: During the assay process, time course images are collected at a fixed time interval (can be anywhere from seconds to 10's of minutes). Prior to each image, a volume of sample (flow solution 1), followed by a volume of detector reagent (flow solution 2), are sequentially passed or flowed over the assay portion of the cartridge using the system's controls and associated flow cartridge. The signal intensities of the assay binding sites (in certain embodiments these will be microarray spots) are extracted from the images and represented numerically as the average of several replicates. Because the sample concentration is kept fixed (through the flowing of unused or fresh solution throughout the assay process) and because the surface concentration of the capture reagent is relatively high (as compared to the analyte/sample concentration), the linear analysis of the time course data yields a slope (used directly as the initial rate in units of signal/time). The initial rate is directly proportional to the amount of antigen presented by the sample (more antigen equals a larger initial rate). In the case of very high antigen concentration that results in either saturation of the imager (PMT out of range) or surface saturation (loss of linearity), the linear fit is made with only earlier time points. In the case of moderate and low concentration, additional or all time points can be used for the linear fit.

In some embodiments, ‘Non-Linear Data’ are produced by the system and methods herein disclosed, and analysed as follows: An alternative to the linear data described above is a method that utilizes a form of acceleration or amplification of signal, typically resulting in the generation of non-linear data. In such a case, the signal is initially dependent on the binding of analyte, as in the linear data case, but then acts as a catalyst for the generation of additional signal from a “set” of accelerator reagents (described in further detail below). Typically, the set of accelerator reagents are comprised of two reagents, one placed in reservoir 1 (tube 1) and the other in reservoir 2 (tube 2), enabling the accelerated signal to be a factor of the number of iterative flow cycles and the amplification properties of the accelerator reagents. The signal verses time data produced, if linear, is fit as above, if non-linear, it is fit to a polynomial type equation, and the first derivative is used to extract the slope at a specified analysis time. Regardless of how long the time course binding experiment precedes, any earlier analysis time may be used for back calculations. Early analysis times typically yield lower sensitivity results and as such are useful for measuring high concentration analytes simultaneously with low concentration analytes using two different analysis times.

Standardization and back calculation. As described above, initial rates are measured for both standards and unknowns. In general, unknown sample concentrations are back calculated from known concentration standards. During the development of the system and methods herein disclosed, it was determined from the empirical analysis of initial rates with varying analyte concentration, that the initial rate obeyed an equilibrium or saturation model where the rate of the binding reaction approached a maximum with increased concentration. While several assay parameters were discovered that did impact the maximum rate, for any given method the maximum rate effectively determines the highest quantifiable dose (as defined later in this section).

For many enzymes, the rate of catalysis or velocity (V), varies with the substrate concentration, in a linear manner at lower concentrations and reaches a maximum at higher concentrations. In 1913 Leonor Michaelis and Maud Menten proposed a simple model to account of these kinetic characteristics and is described in the equation below, known as the Michaelis-Menten model, with Vₘₐₓ representing the maximum rate of catalysis, S representing the substrate concentration and Kₘ known as the Michaelis constant.

Velocity=(Vₘₐₓ*Sⁿ)/(Sⁿ+Kₘⁿ)

While the Michaelis-Menten model was developed to describe an enzyme catalyzed reaction, and the reasoning behind the observation of Vₘₐₓ in such reactions is based upon the formation of the enzyme substrate complex reaching equilibrium or saturation, the model also describes the data generated through the system and methods disclosed herein. One possible explanation for the observation is related to the solid-phase nature of the binding reaction, where the solution phase analyte must first interact with the surface-phase capture reagent before a reaction or binding event can occur. This first step is perhaps “equivalent” to the formation of the enzyme substrate complex in the formal Michaelis-Menten model. This surface association step is in effect at equilibrium or saturation at higher concentration. Several other models relating to reactions reaching saturation or equilibrium were found to be equally useful in the treatment of the data. Indeed, the Michaelis-Menten equation above represents just one type of equation that can be used to fit the initial rate data. Other embodiments use other similar equations. Examples include but are not limited to:

Wagner Model: fit=exp((A+(B*ln(x)))+(C*(ln(x)̂2)))

One Site Saturation Model with nonspecific Binding: fit=(C+((BLmax*x)/(Kd+x)))

Hyperbolic Equilibrium Model: fit=(C+(BLmax*(1−(x/(Kd+x)))))

Eadie-Hofstee Model: fit=(A+((B*x)/((C*(D+1))+x)))

Hyperbolic Model from C: fit=(C+((A*x)/(B+x)))

Michaelis Menten Model: fit=((Vmax*(x̂n))/((x̂n)+(Km̂n)))

Integrated Michaelis Menten Model: fit=((1/Vmax)*((S0−x)+(Km*ln(S0/x))))

Michaelis Menten Steady State Model: fit=(Vmax/(1+(Km/x)))

Michaelis-Menten equation: fit=((BLmax*x)/(Kd+x))

MMF Model: fit=(((A*B)+(C*(x̂D)))/(B+(x̂D)))

as well as many other similar such models known to the art.

It should be noted that the rate equation itself is not novel, these have been used for enzyme type reactions for many years. The key novelty and breakthrough described herein is the use of these equations (enzyme catalysis based reaction equations) to describe an assay (typically a biological assay, and in certain embodiments a protein based biological assay) processed using the system and methods disclosed herein.

Such an approach to the calculation of targeted analyte concentrations from within complex (or simple) samples, facilitates the time based analysis central to the system and methods disclosed herein, which in turn delivers the unique capability of the system of being able to accurated quantify the concentrations of different target analytes from a single sample that are present at vastly different concentrations within that sample. This is because those target analytes present in very high concentrations are detected and measured (concentrations calculated) early in the assay process, whereas those target analytes present in very low concentrations are detected and measured (concentrations calculated) later in the same assay process (when the system detects the binding curve generated from these lower concentration analytes).

This rate based analysis approach also facilitates the systems unique capabilities to discriminate specific from non-specific signals (targeted from non-targeted binding) within an assay as described in further detail below.

### 5. Specific Versus Non-Specific Signals

Differentiating specific signals (signals from the binding of a targeted analyte to a capture agent) from non-specific signals (signals generated by the binding of a non-targeted substance/molecule/analyte to a capture agent) offers significant benefits to many areas of assay development, validation and processing.

The system and methods disclosed herein deliver these benefits. In summary—in order to discriminate non-targeted from targeted binding or to validate a signal as targeted, the sample under test must adhere to the trajectory of the binding rate/curve observed during the standardization process.

Although a slope—and therefore an analyte concentration—can be determined after two measured signal intensities, such a determination is more accurate following measurement of a third or subsequent signal, particularly if the third, fourth, fifth or subsequent signal is of an intensity that is at least twice the standard deviation of the background signal. FIG. 11, for example, shows a plot of signal intensities of an IL-1b antibody (capture agent) binding to a targeted IL-1b antigent (specific analyte), alongside a plot of signal intensities of an IL-1b antibody (capture agent) binding to a streptavidin (representing a simulated non-targeted binding signal). In this example, in addition to being non-linear from the third measurement, the first measured rate is significantly outside the calculated Vₘₐₓ for IL-1b. Each of these observations gives cause to classify the IL-1b antibody to streptavidin signal and associated plot as one resulting from a non-specific (non-targeted) binding event to a binding site (and the capture agents therein).

Alternatively, in a case where a signal is a result of a relatively high concentration of non-specific interactions, the initial rate will be observed to be very high at early time points and approach zero at later time points. In general, however, the distinction between a real and non-real signal comes from the comparative analysis of the binding curve from a given experiment with an unknown analyte presence and concentration, to the binding curve of a standard analyte with a known presence and concentration.

Additional discrimination of specific from non-specific binding events (true or false signals) may be made as follows (noting that for the purposes of a sandwich type assay, a specific signal is one wherein the targeted analyte bridges between the capture agent and detector reagent, whereas a non-specific signal is one wherein some other substance/molecule/analyte, other than the targeted analyte, either bridges between the capture agent and the detector reagent, or sticks directly to the surface of the assay portion of the cartridge and subsequently attaches the detection reagents and/or directly attaches the accelerator reagent, e.g. biotinylated dendrimer, or the streptavidin to the cartridge surface):

Option 1: At the end of the assay run, swap out the sample and detection reagents and replace with assay buffer and assay buffer containing a certain concentration of the capture agent.

Option 2: At the end of the assay run, swap out the sample and detection reagents and replace with assay buffer and assay buffer containing a certain concentration of the detection reagent.

Option 3: At the end of the assay run, swap out the sample and detection reagents and replace with assay buffer and assay buffer containing a certain concentration of the targeted analyte(s).

In each Option detailed above, run a few cycles of iterative flow of each of these reagents and image the assay portion of the cartridge. In the case of specific binding, the signal would decrease, and thus in this instance the signal and associated binding curve can be deduced to be a specific signal from a targeted analyte. However, if the signal is non-specific in nature then the signal should remain mostly stable as it is not dependent upon the presence of the analyte of interest (target analyte); in such circumstances the signal and associated binding curve can be deduced to be a non-specific signal from a non-targeted substance within the sample.

Put another way, because the system disclosed herein detects signals in real time, at the end of the assay run a moderate to high concentration of something specific (as outlined in the above Options) may be inserted into the reservoirs for the cartridge and system, and flowed across the assay portion of the cartridge. If the signal is observed to decrease, this is a further indication that the signal is specific in nature (real and analyte dependent), whereas if the signal is observed to remain broadly constant in its intensity, this is an indication that the signal observed is non-specific (false).

Additional discrimination of specific from non-specific binding events (true or false signals) may be made as follows: In order to measure the disassociation, rather than adding in additional reagents as described above, only remove the analyte or sample and/or detection reagents. The kinetic binding signature subsequently measured will provide further insights into whether the signal is created from a specific or non-specific binding event. A fast and low signal intensity change would be indicative of a non-specfic binding event, whereas a slow and high signal intensity change would be indicative of a specific binding event. This ‘disassociation rate’ difference between non-specific and specific binding mirrors the ‘asossiation rate’ differences described previously (high to low rather than low to high signal intensity development). As the association and disassociation rates may be different, this approach suppliments the previously descibed methods for differentiating specific from non-specific binding events and associated signals.

Yet a further approach and method to differentiate non-specific (non-targeted) binding to specific (target analyte) binding interactions in an assay, and to further adjust and correct signals that are formed through a mix of both specific and non-specific components, relates to the use of the control spots contained in the assay portion of the cartridges. The most common type of background signal is caused by heterophillic antibodies, these are essentially anti-species antibodies which are found in human serum samples, mostly those serum samples coming from auto-immune patients such as rheumatoid arthritis and irritable bowel syndrome and similar such conditions. These heterophillic antibodies are capable of bridging between the capture and detection antibodies, and giving a signal that appears analyte dependent (i.e. is based on the concentration of the targeted analyte), but in truth is not.

By the inclusion of a ‘negative control spot’ that contains a mixture of antibodies (goat, rabbit, mouse, etc.) which are the same species as the capture agents (capture antibodies in this example) used in the assay(s), but which are not directed towards any of the targeted analytes being measured in the assay(s), the system disclosed herein is then able to detect samples which contain heterophillic antibodies, as this ‘negative control spot’ would produce a signal in a sample dependent manner. The signal from this ‘negative control spot’ can thus be used to adjust the signal at the analyte specific spots, to adjust for the “concentration” of heterophillic antibodies present, thus delivering a more accurate measurement of the targeted analyte concentration. Furthermore, samples which contain heterophillic antibodies will be readily identifiable using this novel method, and such samples can be ‘flagged up’ as having a higher level of uncertainty in the quantification process.

### 6. Accelerator Methods

In some embodiments of the invention, through the intentional use of accelerator reagents to increase assay sensitivity and dynamic range, non-linear rate data may be obtained and may be characteristic for a given assay protocol. For example, an accelerator reagent may be implemented which accelerates or amplifies the signal intensity resulting from the antigen-capture agent binding.

Accelerator agents are typically comprised of two reagents and are recursively passed over a bound analyte. The first reagent may interact with the analyte and/or the detector reagent and/or the other accelerator reagent. The second accelerator reagent will interact with the first accelerator reagent to produce a secondary network of accelerator only reagents, whose concentration directly reflects the concentration of the analyte. Examples of these types of reagents include, but are not limited to; Reagent 1: streptavidin, avidin, dye-labelled versions of streptavidin, avidin; Reagent 2: dimeric biotin molecules, PAMAM dedrimers that are partially or fully labelled with biotin, or any biotin containing molecule or macromolecule that can form biotin-avidin networks. More broadly, accelerator agents may include but are not limited to: biotinylated proteins, biotinylated antibodies, biotinylated peptides, biotinylated strands of DNA, biotinylated dendrimers, anti-species antibodies, and any agent capable of bridging between a captured antibody and the detection species in an analyte independent manner.

This acceleration or amplification of signal intensity is thus a function of the number of iterative flows of the first and second liquids (samples and reagents). The resulting data, if non-linear, may be fit to a polynomial equation, the first derivative of which may be used to calculate the slope of the binding curve at a particular timepoint. The key feature being a consistent determination of the initial rate or slope of the sample during standardization and unknown sample analysis. Additionally, a linear portion of the binding curve may be utilized for calculating the rate of binding.

### 7. Typical Assay Parameters

The system and methods disclosed herein enable the processing of assays and the associated generation of assay data. Typical assay parameters used to illustrate and present the data include but are not limited to:

Least Detectable Dose (LDD):

Defined as the lowest concentration of an analyte that can be observed above the background signal with a 60-90% confidence. LDD may be calculated as the initial rate and a subsequent back-calculated concentration associated with a signal that is either one standard deviation above the average background signal or just above the zero dose signal measured. The background signal may be based on the signal obtained from the binding site prior to flow of the analyte sample and/or detection reagent or the signal obtained from an area between binding sites, which typically will represent a very low signal intensity, even after flow of the analyte sample and/or detection reagent. Regardless of the method chosen, the LDD is typically set not to exceed a value that is a given multiple (optionally 3 times) the signal intensity of the lowest non-zero analyte concentration observed. The LDD can be verified by running a known standard concentration at the LDD and calculating a percent recovery.

Least Quantifiable Dose (LQD):

Defined as the lowest concentration of an analyte that can be observed above the background signal with 90-95% confidence. LQD may be calculated as the initial rate and subsequent back-calculated concentration associated with a signal that is either two standard deviations above the background signal or twice the zero dose signal measured, whichever yields the least sensitive LQD. Again, the background signal may be based on the signal obtained from the binding site prior to flow of the analyte sample and/or the detection reagent or from a region not associated with a binding site. The standard deviation of the background signal may be calculated from five signal intensities taken at the end of an assay run. The standard deviation is divided by the total incubation time to determine the initial rate and associated LQD in concentration units. When the zero dose method is used, a value equal to twice the rate observed from the zero dose may be used. Regardless of the method chosen, the LQD is set not to exceed a value that is three times the lowest measured non-zero standard concentration. The LQD can be verified by running a known standard concentration at the LQD and calculating a recovery percentage.

Highest Quantifiable Dose (HQD):

Defined as 5% below a calculated maximum velocity or rate of catalysis, Vmax, (as previously disclosed) but typically not exceeding the highest measured standard concentration. The HQD can be verified by running a known standard concentration at the HQD and calculating a recovery percentage.

Dynamic Range:

The high to low concentration range capable of being quantitated within an assay. Defined by the HQD as the high end and LQD as the low end.

Data Variability/Precision:

The inherent variability of the measured signal intensity for experiments that are performed under essentially the same experimental conditions over varying timeframes. The intra- and inter-experimental variations of analyte quantitation (CV's) are passively acquired through multiple experiments demonstrating LDD, LQD, and HQD. Reagents are held constant to yield spot-to-spot, experiment-to-experiment (intra-day), assay cell to assay cell, cartridge to cartridge, and day-to-day variation

### Example Experimental Designs, Runs and Data

This section summarizes one possible approach to running assays on the system herein disclosed and also incorporates some example data.

Overview:

This example experimental approach is herein referred to as “Real-Time ELISA” or “Real-Time Protein Quantitation”. The Real-Time ELISA is a good description because it uses standard commercially available ELISA kits and reagents that have been reformatted into the system and methods disclosed herein to provide comparative benchmark data, plus complimentary data only available through the system and methods herein disclosed.

Real-Time ELISA Method:

Typical ELISA kits come complete with the following reagents: A) capture antibody (specific to target), B) protein/antigen target (as the known concentration standard), C) labeled secondary antibody (specific to target with biotin label). Typically an ELISA assay involves additional steps (“Enzyme Linked”), where the biotin is further reacted with another enzyme (SA-HRP) and signal developed by the catalytic action of the HRP on a dye substrate.

In the Real-Time ELISA the capture antibody (Reagent A) is printed (arrayed) or otherwise placed onto the flow cartridges herein disclosed. As in the ELISA assay, the Real-Time Method uses the protein/antigen target (Reagent B) as the known concentration standard. Unlike the ELISA assay, the secondary antibody (Reagent C) is used in combination with a selected streptavidin dye reagent (SA-Cy3 or equivalent) to develop the signal from the captured/measured antigen target. In a two flow method (involving the iterative flow of reagents and samples through two flow channels), the antigen target and SA-dye are combined and placed in flow channel 2 (reservoir 2) and the secondary antibody is placed in flow channel 1 (reservoir 1). The recursive and sequential flow of channel 1 and channel 2 over the printed capture antibody generates the real-time signal used to quantitate the antigen target concentration.

### 1. The Experiment

In this embodiment of the system and methods disclosed herein, a triplex assay was used to test for three human analytes—C-reactive protein (CRP), interleukin-1β (IL-1b), and interleukin-6 (IL-6). Although any other analytes and any number thereof may be used, with the three used herein included only as illustrative examples of the assay process, system, methods and device. A study designed to test the efficacy and accuracy of the triplex assay was carried out, with the study design and relevant procedural and system information described below.

Each cartridge contained an assay portion with binding sites for each of the CRP, IL-1b and IL-6 and reservoir portions for containing the test sample and various buffers and reagents employed.

These binding sites comprise capture agents printed or otherwise applied to the assay portion at known concentrations and amounts. In this case, the capture agents were known antibodies for the human CRP, IL-6, and IL-1b analytes. Here, and as would be typical for many embodiments, but not all, the cartridges also included positive and negative controls intended to adduce predictable results when each sample was run. Furthermore, the systems inbuilt control features ensured that the appropriate reagents were added, at approximately the correct concentrations.

The cartridges were first used to prepare standard binding curves for each analyte. In practice, such standard binding curves may be provided or otherwise available. For each analyte, serial dilutions were prepared from a standard of known concentration and analyzed along with a “zero dose” sample containing no analyte. A sample dilution was added to a sample reservoir of a cartridge, with the other required buffers, reagents, etc. contained within separate reservoirs. In some cases, a cartridge may contain a plurality of sample reservoirs, such that samples of different dilutions or different samples may be analyzed using the same cartridge

In creating the triplex standard binding curves, sample detections were carried out in triplicate for each of the eight (seven dilution and one zero dose) concentrations. In this study, a volume of sample containing unlabeled analyte was first flowed through the assay portion of the cartridge from the sample reservoir portion, followed by a volume of detector reagent containing the detection label. A fluorescent detection label was employed in the study but, as noted previousy this is only one illustrative example of an experiment using the system and methods herein disclosed, and other detection labels and corresponding detection devices may be employed.

Following flowing of the detector reagent, a detection device—here, a fluorescence detector—detects the presence of the detection label in the assay portion and a signal intensity is recorded via imaging. As previously described in more detail, as well as in the '044 application, but will be apparent to one skilled in the art, the fluorescence detector includes an exciter, commonly a laser, for exciting the detection label at a particular wavelength. In response to such excitation, the detection label fluoresces at a different wavelength and its signal intensity is measured.

### 2. The Data

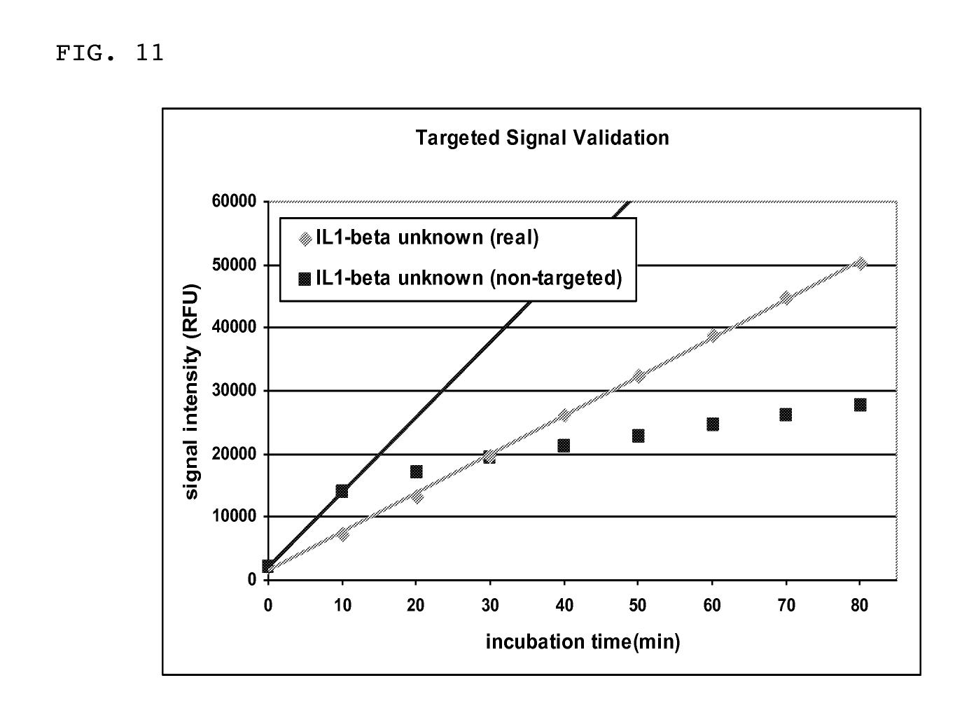

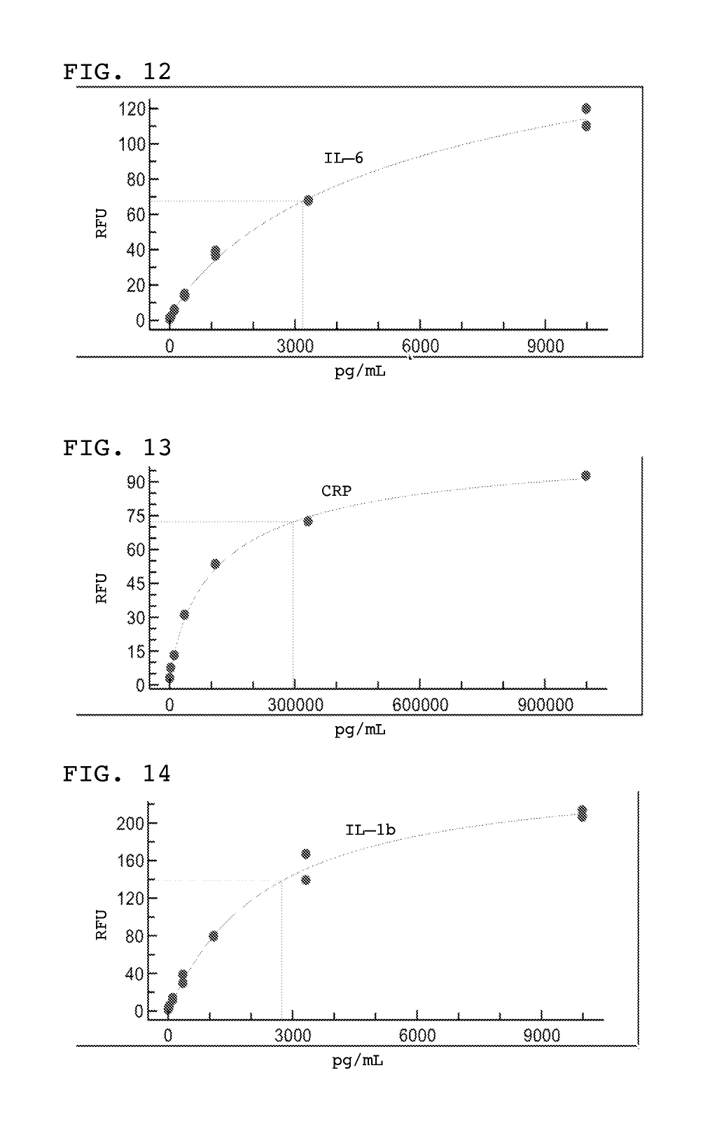

FIGS. 12-14 show plots of the standard binding curves for, respectively, IL-6, CRP, and IL-1b. The binding curves are fit to the individual signal intensities, as shown. The initial rate of each binding curve was calculated from the slope of the linear portion of the resulting binding curve. In the case in which the characteristic binding curve is non-linear a higher order rate equation or derivative of the polynomial at a given time, can be used to determine the overall rate of reaction. For each standard concentration a particular slope (or initial rate) was determined. The subsequent initial rate verses concentration data was fit to an equation that describes enzyme catalysis (a version of which was disclosed earlier). Following standardization an unknown slope (or initial rate) can be back calculated to a concentration based on the rate equation and fit.

Preferably, at least three runs of each concentration standard are used to create a consensus standard curve. The precision of the method can be assessed from the replicate runs and either initial rates, back-calculated concentrations, or recovery percentages. The accuracy of the method can be assessed across the concentration range, looking at back-calculated concentrations or recovery percentage. Table 1 summarizes data from the example experiment.

TABLE 1
(example data from triplex longitudinal assay)

| | LDD (pg/mL) | LQD (pg/mL) | HQD (pg/mL) |
|---|---|---|---|
| IL-6 [symbol] | 3.74 | 8.66 | 229,441 |
| CRP^{†} | 226.23 | 548.01 | 5,314,557 |
| IL-1b^{‡} | 4.11 | 8.11 | 39,407 |

* The zero dose (ZD) calculation method for IL-6 yielded an LDD of 1.60 and an LQD of 3.6, while the standard deviation (SD) method yielded an LDD of 3.74 and an LQD of 8.66.
^{†}ZD LDD = 226.23, ZD LQD = 548.01; SD LDD = 87.04, SD LQD = 213.83.
^{‡}ZD LDD and LQD intersected X-axis; SDD LDD = 4.11, SD LQD = 8.11.

What is claimed is:

1. An assay system comprising:
a cartridge device including:
at least one reservoir portion for holding one or more liquids; and
at least one assay portion for receiving the one or more liquids from the at least one reservoir portion, the at least one assay portion having a plurality of binding sites over which the one or more liquids can be repeatedly flowed; and
a measurement device for measuring binding of one or more analytes in the one or more liquids to the plurality of binding sites.

2. The assay system of claim 1, further comprising:
an interface into which the cartridge device is received and removed, wherein the interface includes an apparatus for controlling flow or movement of the one or more liquids from the at least one reservoir portion through the at least one assay portion,
wherein the apparatus for controlling flow of the one or more liquids can independently control at least one of the following:
the rate of flow of the at least one liquid across the at least one assay portion,
the duration of flow of the at least one liquid across the at least one assay portion, or
the number of times a quantity of the at least one liquid is flowed over the at least one assay portion.

3. The assay system of claim 2 further comprising:
a flow rate sensor,
wherein the interface provides for either or both of variable positive pressure or variable negative pressure to control at least one of a flow rate or a flow duration of the one or more liquids across the one or more assay portions based on a reading from the flow rate sensor.

4. The assay system of claim 1, wherein:
the one or more liquids includes at least one label selected from a group consisting of: a fluorescent label, a luminescent label, and a colorimetric label; and
the measurement apparatus is selected from a group consisting of: a fluorescent measurement apparatus, a luminescent measurement apparatus, and a colorimetric measurement apparatus.

5. The assay system of claim 4, wherein:
the measurement device is configured in the system such that under computer control, a laser or other form of label stimulation can be directed onto the at least one assay portion of the cartridge and subsequent detection and/or measurement of the fluorescent, luminescent or colorimetric signals can be performed.

6. The assay system of claim 1, further comprising:
computer software, which, when executed, is operable to:
analyze a representation of one or more binding curves of the one or more analytes binding to one or more of the plurality of binding sites in the one or more assay portions;
compare the analysis to one or more known standard time course binding curves for the one or more analytes; and
determine at least one of a presence or a concentration of the one or more analytes in the one or more liquids.

7. The system of claim 1, wherein the at least one reservoir portion is covered by a thin membrane that seals a liquid in the at least one reservoir portion.

8. The system of claim 1, wherein the one of the liquids contains an accelerator molecule or entity that provides at least one additional binding site for a detector reagent.

9. The system of claim 8, wherein the accelerator molecules or entities is selected from a group consisting: streptavidin, avidin, dye-labelled versions of streptavidin, avidin; dimertic biotin molecules, PAMAM dedrimers that are partially or fully labelled with biotin, or any biotin containing molecule or macromolecule that can form biotin-avidin networks, biotinylated proteins, biotinylated antibodies, biotinylated peptides, biotinylated strands of DNA, biotinylated dendrimers, anti-species antibodies, and an agent capable of bridging between a captured agent and the detection reagent in an analyte independent manner.

10. The system of claim 1, wherein at least one of the plurality of binding sites contains one or more of the following: a biological entity or a chemical entity.

11. The system of claim 10, wherein:
the biological entity is selected from a group consisting of: proteins, hormones, antibodies, antigens, viruses, antibody complexes, antibody fragments, peptides, cells, cell fragments, aptamers, cell lystates, fractionated cell lysates, fractionated cells, DNA, RNA, mRNA, genes, and genetic expression products; and
the chemical entity is selected from a group consisting of: chemical elements, chemical compounds, pharmaceutically-active compounds or their metabolites, minerals, and pollutants.

12. A method of calculating an analyte concentration using the system of claim 1, the method comprising:
making a plurality of time-sequenced measurements of a signal from the plurality of binding sites;
creating a kinetic binding curve using the plurality of time-sequenced measurements;
calculating a slope of the kinetic binding curve, wherein the slope of the kinetic binding curve is representative of a binding rate; and
comparing the binding rate to rate-based binding curves for known standards for the analyte.

13. The method of claim 12, wherein creating the kinetic binding curve includes plotting the plurality of time-sequenced measurements as a function of cumulative duration of interaction of the one or more liquids and a capture agent within at least one of the plurality of binding sites.

14. The method of claim 13, further comprising:
calculating at least one first derivative of the plotted plurality of time-sequenced measurements, each of the at least one first derivatives calculated using an adjacently-plotted measurement.

15. The method of claim 12, wherein a shape of the kinetic binding curve is used to distinguish specific binding of the analyte to the capture agent from a non-specific interaction of the capture agent to a non-targeted analyte.

16. The method of claim 15, further comprising:
targeting for drug development, drug discovery, or diagnostic use those analytes exhibiting specific binding.

17. A method of distinguishing specific binding and non-specific binding in an assay, the method comprising:
obtaining a plurality of signal intensity measurements of an analyzed sample, each of the plurality of signal intensity measurements being made during or following an interaction of the analyzed sample and a capture agent for a targeted analyte within the analyzed sample;
plotting the plurality of signal intensity measurements as a function of cumulative duration of interaction of the analyzed sample and the capture agent; and
in the case that the plotted signal intensity measurements are characteristic of the known standard, determining that signal intensity measurements are indicative of specific binding of the targeted analyte and the capture agent.

18. A cartridge device comprising:
at least one reservoir portion for holding one or more fluids; and
at least one assay portion for receiving the one or more fluids from the at least one reservoir portion, the at least one assay portion having a plurality of binding sites over which the fluid is flowed, and being connected to the at least one reservoir portion through fluidic channels or tubing.
